SEQUENCE LISTING (5 sequences)

SEQ ID NO: 1 - protein, 151 aa, Homo sapiens
MERPEPELIRQSWRAVSRSPLEHGTVLFARLFALEPDLLPLFQYNCRQFSSPEDCLSSPEFLDHIRKVMLVIDAAVTNVEDLSSLEEYLASLGRKHRAVGVKLSSFSTVGESLLYMLEKCLGPAFTPATRAAWSQLYGAVVQAMSRGWDGE

SEQ ID NO: 2 - protein, 151 aa, Mus musculus
MERPESELIRQSWRVVSRSPLEHGTVLFARLFALEPSLLPLFQYNGRQFSSPEDCLSSPEFLDHIRKVMLVIDAAVTNVEDLSSLEEYLTSLGRKHRAVGVRLSSFSTVGESLLYMLEKCLGPDFTPATRTAWSRLYGAVVQAMSRGWDGE

SEQ ID NO: 3 - DNA, 518 bp, Homo sapiens
  CDS (1)..(456)
atggagcgcccggagcccgagctgatccggcagagctggcgggcagtgagccgcagcccgctggagcacggcaccgtcctgtttgccaggctgtttgccctggagcctgacctgctgcccctcttccagtacaactgccgccagttctccagcccagaggactgtctctcctcgcctgagttcctggaccacatcaggaaggtgatgctcgtgattgatgctgcagtgaccaatgtggaagacctgtcctcactggaggagtaccttgccagcctgggcaggaagcaccgggcagtgggtgtgaagctcagctccttctcgacagtgggtgagtctctgctctacatgctggagaagtgtctgggccctgccttcacaccagccacacgggctgcctggagccaactctacggggccgtagtgcaggccatgagtcgaggctgggatggcgagtaagaggcgaccccgcccggcagcccccatccatctgtgtctgtctgttggcctgtatctgttgt

SEQ ID NO: 4 - DNA, 626 bp, Mus musculus
  CDS (77)..(532)
gctgcatgtgcgttgactgcacccacgcctcgagggtcccatcactgcgtcccgcgagtctcctgggagagagagcatggagcgcccggagtcagagctgatccggcagagctggcgggtagtgagccgcagccctctggaacatggcactgtcctgttcgccaggctcttcgccctggaacccagcctgctgcctctcttccagtacaatggccgccagttctccagccctgaggactgtctctcctctccagaattcctggaccacattaggaaggtgatgctagtgattgatgctgcagtgaccaacgtggaggacctgtcttcattggaggagtacctgaccagcttgggcaggaagcatcgggcagtgggagtgaggctcagctccttctcgacagtaggcgagtccctgctctacatgctggagaagtgcctgggtcccgactttacaccagctacaaggaccgcctggagccgactctacggagctgtggtgcaagccatgagccgaggctgggatggggagtaagagacgagccagtgcccctatctatgtgtgtctgtctgttgatctgcctgttgtagtcttagcctctcccccagggtctctctataccttggtc

SEQ ID NO: 5 - DNA, 7727 bp, Homo sapiens
  exon (2646)..(2734)
  exon (4200)..(4311)
  exon (4941)..(5060)
  exon (6856)..(6990)
ctagttacttaacctctttgcctccatttcctcagctgtaaaacgagggtatgataaatagcacctacttcgtagttattgtgaggatgaaaagaggaaatgaagtaaggtgcccggaacactgtctagacataaatgccagctctgtattattaatgatacatgaacctcaatgaccagcactggctctgggacagtacttgaattctcctctaggttcttacctcctgggcggatgacccacagggaggagactcctgtgacttttactgagcccctcaaatgtgggcttggacccttggtctactgatgggaatacatcactctcatcgcttaggcccatccagattctcaggcctttcttgaagggagcagggatagagtgcacttaccttggcttatcttagcacatatctcctggctctaggaagacaaagagtgaagccatggtttgagtactgactctacttactagctagggccgttaactcccactacctcatcagactccagaccctggccagaggagagaggaagcaagatgggtcctaagtatagattgctggcaagaagcgtgagctgggtagagtggggtacctgagaccagccaggtggcctgcacagagcactgagcatctgggcactgaatagtgaaggtcaccaatgacacgtgtccttgccctgtgacctgctgcacctggctgcaggtccacgggaccccaagctggaagtttagtcacgtcttgttccttcagccccaatcactttgtgttcttgcttggagacctcttggaccctaggacaccagcttttccaggctgccttatagccttgctctcctgctccagcccagagatttgtgaggaaatgggaaatagaataggggttaccacaaactcctgagtccttggaagagggagaacagctagatgatttcctggggcatggctaggagtaggaaagaaaagttctgctgaggcactcagttctgattcagggccagaaggagaaattttaaagaccaggactcgccactccatgtctagcacagggctgggcatggagccccagctgttcataaccaggggctcaagaacacccaccttctacccatggaactaaaggaggtggactaaaaggtcccagagacctcaccacccaacccctttgctgcacaactgaggaaacagggccagagaggcgtggacagacagaggagataggctcggagacccctgagggaataatgagagagaaataaaggtgaaaacgggcttgctctcccaaacctgggccaccagcactgggctggggcttgaggaactttctgcttgaaagagaaaagtttagttgtaattggcaggtggccaattgcacaccctgagtggggagcatggattctattatctgaacttctattgaaaaaattaaacagtacttccgttaaaaaaaaaaaaaaaaaaggccgggcgcagtggttcacgcctgtaatccccagcactttgggaggccgaggtgggcggatcatgaggtcaggagatcgagaccatcctgtctaacacggtgaaaccccgtctctactcaaaatacaaaaaatcagcctggcgtggtggcgggcgcctgtagtcccagctactcgggaggctgaggcaggagaatggcgtgaacccgggaggcggagcttgcagtgagccgagatcacgctattgcactccagcctgggcgacagagtgagactccgtctcaaacaaacaaacaaaaagatgaaaaatcatggaagtggggaaggcccagggagggaagaggtgaaaaattaaaattaaaattaaaattaaatcatggaacggtagaatgttagagctgaactccctcaagattccaccctggccaagcagagctgggattagaactggatctcacggctttctaaagatggagtctcaaaaaaacccaggcctggctgggaatggctgtggtcttggccctgtccctacggctgaaagccctggtggcccccaacgtgtgctggtgccctagtcagaccgagaactaggcccctaggccgcctccccaggcctgacctgcaggccaggtgggtggcaaactcaaatgcctctggcaatgcgtgagttgaggggcctgtggagagggggcgcatgcagcgcctacctgaggcgaccaaattcaacacagtgccctcggggctcgggaggagaaactgaggcccagagagcctccgcgatgtcagttacgttctgcaggccgggcctttcccaggccaccatagcggctggcggagggagcgcgcgccttgctggcctggagggggcgggggccgtggcggctttaaagcgcccagcccaggcgtcgcggggtggggcggctctggcggctgcggggcgcagggcgcagcggccaagcggggtccccggaagcacagctggggtgtctccacctacgactggccgcgcgccttttctctcccgcgccagggaaggagcggctgcggcccccgccgggcggaggcacggggggcgtacgaggggcggaggggaccgcgtcgcggaggagatggcgcggcacgtgcggtgacggcacccgagccctgagggtcccagccccgcgctccgcgtccccgggacagcatggagcgcccggagcccgagctgatccggcagagctggcgggcagtgagccgcagcccgctggagcacggcaccgtcctgtttgccaggtgagggctacacgagcgcccggggatgcgggtggaggctgcctcggcgggaaaggggtcacgcggcaggaccggccccgaaggagggaaaactggccgctgctggggcgctggggcgcctgccagatgtgcgccagaggagcggggcgcggcgacgggtggcacagcccctctgcctctctctcgaccgtgggcgcctgttgccaaccagcgccgcacaccactctcccagcggggctcaccgggccgggcgggatcccctcccgcggatctccccacatctgggcgggtggacctccgtccctgcgcggcgcctccaccccgccttgtgcctcctgcgtcttccttaggtagcgactgcaggggtgcaccccgctgccggtatcagggtactggggtgccccgagagccggggaagggagatgtgtggggcgagctcctgcgcccgcgtgtgagcggtctggctccaggtgtgtggatccctgacaaccaacaagacaaacctttccgaggaaagagccagagagcagccggggaaggggtgacagatgcgggagcctcaccgcaggggagaggggtggggggtgtgcaggccgggtgggaagggaaggccagtctccaacctcagctcgcagttttggtaacccttccgctatcaccaaccgggcactgcttccctcaccgtggtgttacctatccttggcacccttggacagtccagaacttctacccaggcttccgcaaaaccacccccaagacaaaaggggaaaacattcagttacccggtgagacctcccaaatgccgaggagggcctctgcttctgcctccggactccccccagctcacttttttctcaggcacccgctgtggctgcactacatccaagctgtgtgaccttaggcgagttctagcctccccgggcttctgttttctcctctataaaactgggataacagttgtacccactgcttaggacagtgaggaatacagttaaaagttaaccacacggaaagtactcagcgcaggagggggggggcggggggagagggagagggagagagagagagagagagagagagagagagagagagagagagagagagagagagagagagagagagaaagggctttcctctccacacaacgtaactccaggtttcctgtttggggacctcttcctgtcccaaatcacacccctcagctattcaccgagggacccctgcttaccagaggccctgggacacatttgccctttctaggatgctgcccagagccagtagaaggcatgaggaggagggcactaccgaagctggcgggggagcagtgccaggtgggagagtcagctgtggtttgcagctgagacctggcttggcacccccgcccgctgactgcaggacagccaggcttgctggggcttgcaggagctcacaccccagggctaacacctgggcctcactcccacaggctgtttgccctggagcctgacctgctgcccctcttccagtacaactgccgccagttctccagcccagaggactgtctctcctcgcctgagttcctggaccacatcaggaaggtgagagggaggcagagctgctgtgacctctgggatccagcagagcacctccctggtggagaatggagaaaatgactttctgccccaggtctagtcttactcccttccagttcccccaagcagggtacagctcctttccctctgggctggattggtctgggacatcagaatcagcctcttcccctaccctgcgttcagagagctggtcccagcctgtgtcattgcctttgtcctctggctcccataggttgtgttagcacagatcctggtaattgggaactatctgcctatgaaaatcaggctgagattttcttagaatctcatgattctcaaagggcctcagtttctccttgtcccagcagacaacactctccagtaggtgtgtttagagatgtgaatttctcagaggagagaggcttccttcatgggtataacattagtagtttggccaccacgtatcgggtgccccctacatgtagtagacacaggctgcctttgacccctgatcttggagaactgagggtcctactggcctctcttgctttgggaccctcatgccttgggctgtgattgtgagcagatggggctgctctggcctctctgtttcactgctgccttggccttggcaggtgatgctcgtgattgatgctgcagtgaccaatgtggaagacctgtcctcactggaggagtaccttgccagcctgggcaggaagcaccgggcagtgggtgtgaagctcagctccttctcggtgggtaaaggagctctgactctctccagagctcctggcctggggccacttctctctcccagacctccccttgccacctgttctctctcccattcccatatccttagataaccagggctgcgggcctgcatctgttacctgctgtgtggccctgacatgggcttgacctctctgagcttccaagttctcttttctgatatggcttcttttaaaaccataaaaattaacaagcttttgggatcagctggtctgctcaattttctattgctatgtatcaaacaaccctagaactatgacaatgctttccgatttctcatggctgggctcagctgggcagctcttctgctccacatggtgtagctggagttacttgtgtgttatgtttagctgggagctcaactgaggctagaatgtccaaggcctgtcagcctttgagctctgtccctgtgatttgttgtcattcactagtctagctgagcttctttatagaatagcagctgagttccaagggggagtttttagaggacaagccccagtgtgaaagtgcctataaagcctctgcttgcatcacttttgctaatgtcccattggccaaagccactcacatggataagcccagtgtcaatgtgggaggaccctactcaagggcatgaatccttgaggtgtagcccactgggggcgactgatatgacagtacaccacacagtccagtcaccttgccagatggtgagcaccttgaggaaggagctgtgttggtcttgtcctcctctccatccccagcaccagcatagtgcctgatctaccagggctacagtggatattcagttaatatttgtgagataaataaaccttattttattatgagtaataatggcaaatgagagccattatctttatttatttatttacttacttatttattctggggacagaatcttgctctgtcgcctaggctggagtacagtgatgtgatcacagctcactgcagcctcaacctcccaggctcaagtgatcctcccacctcagcctccctcatagctgggactacaggcatgcaccaccatgcccagctaatttttgtatttttggtagagatggagttttcccatgtttaccaggctggtctcaaactcctgggctcaagtggtctgcccacctcagcctcctaaagtgctgggattataggcgtgagctactgcacctggcctattatcattgtaggtttcctgtatacagatgctgtgctaagagctttttctggattatcccatttaatcatcccaacaggcccaggagttggagcttctactttccctgtatcactgatgaagcagctgaggctcagagaggttacataacttacccaaggtcacccagctgatgaagggtggagtcagaatctgtactcaggtctatctggctccagagcccaggctcttaaccccatcattgctgccttgcagatgaggaaactcaagtctgggagcaatgacttggtttggtctaagctgggaggtcccaggcaatgtcttggccccactcatggggtggttgcaagaccaagacaggggtgaaaaatgtgaggatataaagcctcacatttagagggaaaggtagacacaatgtgatacccaaggatttgggtaggaccctgctcaggaagcatggactcaatgcaggagaggaaaaaaggcccaggagccctaatcagtcctaggcctcagttttctcacctgtggaatggagcagtgcctttctgcctttggaagtgacaaggtgccccttgcctctgcagacagtgggtgagtctctgctctacatgctggagaagtgtctgggccctgccttcacaccagccacacgggctgcctggagccaactctacggggccgtagtgcaggccatgagtcgaggctgggatggcgagtaagaggcgaccccgcccggcagcccccatccatctgtgtctgtctgttggcctgtatctgttgtagcccaggctccccaagcttccctgcatcttggtccttgtccccttggccacactggagaggtgatggggcagggctgggtctcagtatcctagagtccagctgcagaaggagtggcttttcctccaggaaggggcttctgggtgtcccctcatccccagtagcctctttcttgcgtttctttttaccttttttggcactccctctgaccccgcgatgagtgttttggtggcagaggtgggatgagctggaaaggtatggaggtgggagaggatggggctcttctgtctgtcctgcttcttcaggtgagtgcaggccaaggcgggggtgagatggctgagcttccagcgccttctgtcctgcctgcccagtcccttcactgctttcctgccccaagatggcttgcttttcacaaataaagagaaagagcagctttagccttcttggtggaatcccaggcagtgggagcagaatcagaactgccagggaagggaagggggacctgggtctcaatgggtctcatttgagtctcgcgggctgtgcagatgccctgacagagtcggtttcctttggcggcattccctttccctcattcagcacttctgctgggaactccctgactattccgctgctgcaggaacccagctagctggccaggtggggaggggctggggac

(19) United States
(12) Patent Application Publication
(10) Pub. No.: US 2003/0134387 A1
(43) Pub. Date: Jul. 17, 2003

(54) Vertebrate globin

(76) Inventors: Thorsten Burmester, Mainz (DE); Thomas Hankeln, Budenheim (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/204,925

(22) PCT Filed: Feb. 19, 2001
(86) PCT No.: PCT/EP01/01830

(51) Int. Cl. C07K 14/795; C12Q 1/68; C07H 21/04; C12P 21/02; C12N 5/06

(52) U.S. Cl. 435/69.1; 435/320.1; 435/325; 536/23.5; 530/386; 435/6

(57) ABSTRACT

The present invention concerns a neuroglobin, a nucleic acid coding for such a protein and a method for the preparation of such a protein. The invention in addition concerns antibodies directed against the protein and the use of the DNA and the protein for diagnosis and/or treatment of diseases of the nervous system.

The present invention concerns a new vertebrate globin, a DNA coding for such a protein and a process for the manufacture of such a protein. The invention in addition concerns antibodies directed against the protein and the use of the DNA and the protein for the diagnosis and/or treatment of diseases of the nervous system.

Globins are porphyrin-containing proteins which reversibly bind oxygen. Bacteria, plants, fungi and animals have globins. Up to now only two different types of globins have been described in humans and other vertebrates: the heterotetrameric hemoglobins and the monomeric myoglobins. Both of these regulate the transport and storage of oxygen, hemoglobin in the blood and myoglobin in the muscles. Although globins are one of the best investigated proteins and most diverse variants of the two groups are known, no additional globin families have previously been described for vertebrates.

Surprisingly a search in expressed-sequence tag (EST) data banks of the mouse and humans showed that some partial ESTs which are derived from neuronal tissue contain globin-like sequences which do not belong to the family of vertebrate hemoglobins or myoglobins and thus code for a new globin. This new globin is preferentially expressed in neuronal tissue and reversibly binds oxygen with an affinity similar to that of myoglobin.

Diseases of the nervous system such as stroke, Alzheimer's disease, Parkinson's syndrome, dementia, other neurodegenerative diseases and tumors have a variety of etiologies.

However, in any case the maintenance of oxygen supply to the nervous system is essential for the survival and function of neuronal cells. For a number of neurodegenerative diseases oxygen deficiency and a disorder of energy production is considered to be a possible risk factor (e.g. Alzheimer's), or it is at least involved in the complex pathological process (e.g. morbus Parkinson). There is a direct relationship between oxygen deprivation and brain damage in stroke when primary oxygen-deficient, dying cells damage neighboring tissue (ischemic cascade).

the amino acid sequence of SEQ ID No. 1 or 2 or an amino acid sequence which differs therefrom by one or more amino acids. These possible differences can preferably be characterized in that the DNA of the latter amino acid sequence hybridizes with the DNA of SEQ ID No. 3 or 4.

The term “amino acid sequence which differs by one or more amino acids” comprises any amino acid coding for a neuroglobin whose DNA sequence hybridizes with the DNA of SEQ ID No. 3 or 4 and codes for a protein which binds oxygen. The DNA sequence can differ from the DNA of SEQ ID No. 3 or 4 by additions, deletions, substitutions and/or inversions of one or more base pairs. The term “hybridization” refers to a hybridization under the usual conditions, in particular at 25 K under the melting point of the sequence.

Another subject matter of the invention is a neuroglobin gene, in particular a human neuroglobin gene or a gene which differs therefrom in one or more base pairs provided it expresses the function of the neuroglobin.

Another subject matter of the invention is a nucleic acid which codes for neuroglobin. The nucleic acid can be an RNA or a DNA, e.g., a cDNA. A DNA is preferred which comprises the following:

(a) The DNA of SEQ ID No. 3 or a DNA which differs therefrom by one or more base pairs where the latter DNA hybridizes with the DNA of SEQ ID No. 3, or

(b) a DNA which is related to the DNA of (a) via the degenerate genetic code.

(c) The DNA of SEQ ID No. 4 or a DNA which differs therefrom by one or more base pairs where the latter DNA hybridizes with the DNA of SEQ ID No. 4, or

(d) a DNA which is related to the DNA of (c) via the degenerate genetic code.

The DNA of SEQ ID No. 3 was deposited as the E. coli clone phumNGB-1 at the DSMZ (“Deutsche Sammlung von Mikroorganismen und Zellkulturen”) as DSM 13213 on Dec. 22, 1999.

The term “DNA which differs by one or more base pairs” comprises any nucleic acid coding for a neuroglobin which hybridizes with the DNA of SEQ ID No. 3 or 4 and codes for a protein which binds oxygen. The nucleic acid can differ from the DNA of SEQ ID No. 3 or 4 by additions, deletions, substitutions and/or inversions of one or more base pairs. Reference is made to the previous statements with regard to the term “hybridization”.

A nucleic acid according to the invention can be present as such or in combination with any other nucleic acids. In particular a DNA according to the invention coding for a neuroglobin can be present in an expression vector which is also a subject matter of the invention. Examples of such expression vectors according to the invention are known to a person skilled in the art. In the case of an expression vector for E. coli these are for example pGEMEX, pUC derivatives, pGEX-2T, pET3b and pQE-8. pY100 and Ycpad1 are to be mentioned as examples for expression in yeast, whereas pKCR, pEFBOS, cDM8 and pCEV4 are to be mentioned for expression in animal cells. The Baculovirus expression vector pAcSGHisNT-A is particularly suitable for expression in insect cells.

A person skilled in the art knows suitable host cells for expressing the nucleic acid according to the invention present in an expression vector. Examples of such cells comprise the E. coli strains HB11, DH1, X1776, JM101, JM109, BL21 and SG 13009, the yeast strain Saccharomyces cerevisiae or Schizosaccharomyces pombe and the animal cells L, NIH 3T3, FM3A, CHO, COS, Vero and HeLa and the insect cells Sf9. Integration of an expression vector according to the invention into such cells leads to a host cell which is also a subject matter of the invention. A further subject matter of the invention is a process for producing the neuroglobin in which the host cells according to the invention are cultured under suitable conditions.

A person skilled in the art knows in which manner the nucleic acid according to the invention has to be inserted into an expression vector in order to obtain an expression vector according to the invention. He also knows that this nucleic acid can be inserted in combination with a nucleic acid coding for another protein or peptide such that the DNA according to the invention can be expressed in the form of a fusion protein.

Furthermore a person skilled in the art knows conditions for culturing transformed or transfected cells. He also knows methods for isolating and purifying the protein or fusion protein expressed by the nucleic acid according to the invention.

Yet a further subject matter of the present invention are ribozymes which are complementary to the neuroglobin gene according to the invention or to nucleic acids according to the invention and bind to the nucleic acids or to an mRNA transcript of the gene and can cleave them resulting in a reduction or inhibition of the synthesis of the protein coded by the gene or nucleic acid.

The invention is also directed to an antisense RNA which is complementary to a nucleic acid according to the invention which also includes the neuroglobin gene and can bind to this nucleic acid leading to a reduction or inhibition of the synthesis of the protein coded by this nucleic acid.

These means according to the invention provide the basis for measures which can regulate neuroglobin metabolism in order to treat corresponding diseases or specifically study them.

Another subject matter of the present invention is an antibody directed against a protein or fusion protein described above. Such an antibody can be produced by conventional methods. It can be polyclonal or monoclonal. It is preferable to produce it by immunizing animals with an above-mentioned (fusion) protein or fragments thereof which may be advantageously coupled to a carrier molecule such as KLH or BSA; in which rabbits or chicken are used for a polyclonal antibody and mice are used for a monoclonal antibody. The animals can receive additional “boosters” containing the same (fusion) protein or fragments thereof. The polyclonal antibody can then be obtained from the serum or egg yolk of the animals. Spleen cells of the animals are fused with myeloma cells in order to obtain the monoclonal antibody.

Another subject matter of the invention is a drug or a pharmaceutical preparation containing the drug which contains one or more of the following components:

a) at least one ribozyme according to the invention,

b) at least one antisense RNA according to the invention,

c) at least one expression vector according to the invention,

d) at least one neuroglobin according to the invention or a protein having its biological activity,

e) at least one antibody according to the invention and/or a fragment thereof and optionally suitable pharmaceutical auxiliary substances and carriers.

The pharmaceutical preparations according to the invention can also contain additional therapeutic substances.

The drugs or pharmaceutical preparations according to the invention can be used to treat diseases of the (central) nervous system, in particular Alzheimer's, Parkinson's syndrome or dementia as well as other neurodegenerative diseases such as stroke, neuronal oxygen deficiency or tumours in neuronal tissue.

The invention is also directed towards a method in which a sample is contacted with one of the above components a), b) or e) or a nucleic acid according to the invention. A sample in the sense of the present invention is any organic material which can be tested for the presence of a neuroglobin or its DNA or mRNA, for example tissue specimens such as neuronal or other tissues, for example cells from neuronal tissue such as neurons or cells surrounding neurons as well as lysates or extracts thereof or protein or nucleic acid purifications.

Another subject matter of the present invention is a kit. Such a kit contains one or more of the following components:

a) at least one DNA according to the invention,

b) at least one neuroglobin according to the invention,

c) at least one antibody according to the invention, as well as

d) common auxiliary substances such as carriers, buffers, solvents, controls, etc.

In each case one or several representatives of the individual components can be present. With regard to the individual terms reference is made to the explanations given above. These apply here correspondingly.

Another subject matter of the present invention is the use of the neuroglobin, the nucleic acid or the antibody as stated above for identification or design of a binding partner.

Finally the invention is directed to the use of the neuroglobin, the nucleic acid, the ribozyme, the antisense RNA or/and the antibody as stated above.

The present invention enables an etiological investigation of diseases of the nervous system. Neuroglobin can be detected by using an antibody according to the invention. A relationship can be made between neuroglobin and a disease of the nervous system. Furthermore neuroglobin can be used to detect autoantibodies directed against this protein. Both tests can be carried out by conventional methods in particular by a Western blot, an ELISA, an immunoprecipitation or by immuno-fluorescence. Furthermore a nucleic acid according to the invention and in particular a DNA and primers derived therefrom, can be used to detect the organisation and expression of the gene coding for neuroglobin. This detection can be carried out in the usual manner, in particular by sequencing, by a Southern or Northern blot or by means of in situ hybridization or by means of RT-PCR.

Furthermore, the present invention is suitable for taking measures against an excess or deficit of neuroglobin in persons. Neuroglobin can be inhibited by an antibody according to the invention. The expression of the DNA coding for neuroglobin can also be inhibited by using a nucleic acid according to the invention, in particular a DNA, as the basis for producing antisense RNA. This can be introduced in persons or certain tissues, in particular tumors, as such or in the form of an expression vector in which case the vector can contain an inducible promoter. Moreover the expression of neuroglobin can be amplified by additionally introducing a nucleic acid according to the invention. This can be introduced in persons or certain tissues, in particular tumors, as such or in the form of an expressing vector in which case the vector can contain an inducible promoter. Moreover neuroglobin can be used as a basis for developing chemical compounds which inhibit or increase the activity of neuroglobin.

Hence the present invention provides means for improved diagnosis of diseases of the nervous system and for treating these diseases.

### BRIEF DESCRIPTION OF THE FIGURES

FIG. 1 shows the genomic organisation of the human neuroglobin gene. Exons are shown by vertical bars and the length of the exons or introns is given in base pairs. The positions of the translation start codon (ATG) and the stop codon are shown. The lower part shows the position and type of repetitive DNA sequences which were identified in the analysed region.

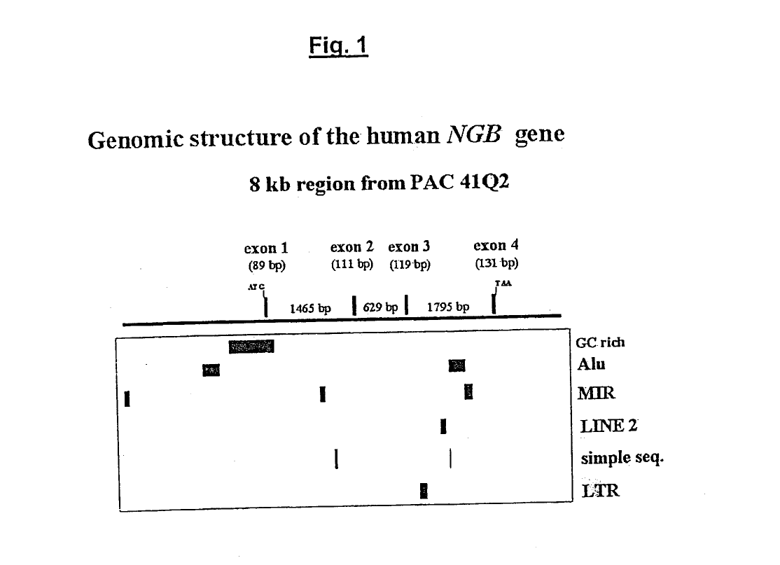

FIG. 2 shows a sequence alignment and the domains in a comparison of human and murine neuroglobin (HsaNGB and MmuNgb) with human and murine myoglobin (HsaMB, accession number M14603; MmuMb, P04247) and hemoglobin alpha and beta (HsaHBA, J00153; HsaHBB, M36640; MmuHba, A45964); MmuHbb, P02088). The globin consensus numbering is shown under the sequences. The secondary structure of human hemoglobin P is shown above the uppermost row. The alpha helices are denoted A to H. The grey shaded amino acids are conserved between the neuroglobins and myoglobins or hemoglobins. The positions of the introns in the genomic human neuroglobin (B12.2, El1-0 and G7-0) are indicated by arrows.

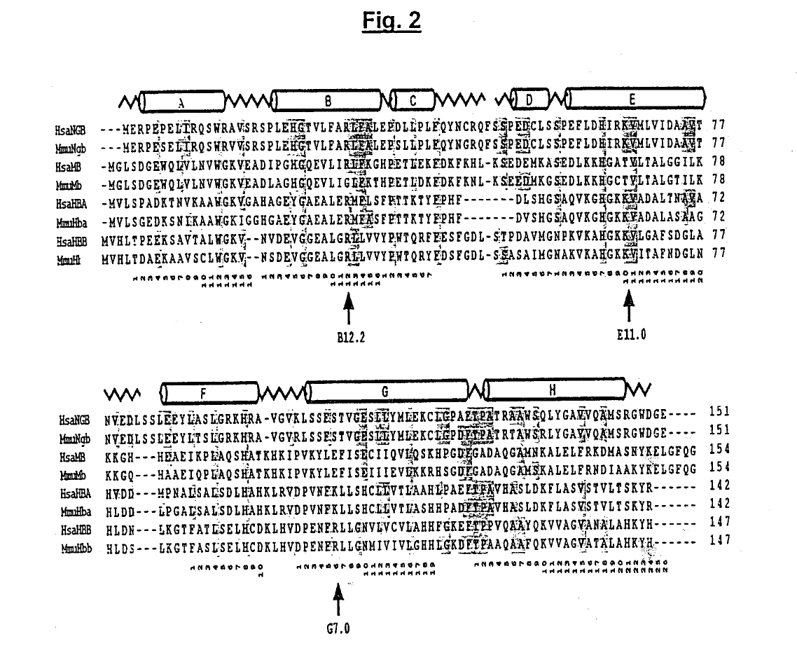

FIG. 3 shows the detection of neuroglobin sequences in normal tissues or neuronal tissue by ern dot blotting.

FIG. 4 shows the oxygen binding of recombinant murine neuroglobin. The figure shows the absorption spectra of recombinant oxy and deoxy neuroglobin of the mouse.

The present invention is elucidated by the following examples.

### EXAMPLE 1

### Identification and Cloning of Neuroglobin

The data banks (available under http://www.ncbi.nlm.nih.gov) of the human and murine EST sequences (expressed sequence tags; Boguski et al., 1993) were analysed with the aid of the BLAST algorithm (Altschul, S. F., Madden. T. L., Schäffer, A. A., Zhang, J., Zhang, Z., Miller, W. and Lipman, D. J. (1997) Gapped BLAST and PSI-BLAST: a new generation of protein database search programs. Nucl. Acids Res. 25, 3389-3402) in order to identify previously unknown globin homologues. Using various protein sequences of invertebrate globins it was possible to identify several, incomplete EST sequences in the mouse as well as humans on the basis of the BLOSUM 45 substitution matrix (non-standard setting; suitable for examining only remotely related sequences; gap existence cost 14, per residue gap cost 2, lambda ratio 0.87) which exhibit significant sequence similarities to globins but which belong neither to hemoglobins nor to myoglobins of these organisms or other vertebrates.

Specific oligonucleotide primers were prepared on the basis of partially present EST sequences. The neuroglobin cDNAs of humans and mice were subsequently amplified with the aid of the RT-PCR technique from the brain RNA of humans and mice. The cDNA fragments were sequenced by a modified chain termination method using fluorescent-labelled dideoxynucleotides. A filter with ordered PAC clones of human DNA was hybridized with the radioactive neuroglobin cDNA probe in order to identify the human neuroglobin gene.

The neuroglobin gene which is completely contained in the PAC clone RPCIP7040021 141Q2 was sequenced using a combined shotgun/primer-walking method.

### EXAMPLE 2

### Detection of a Neuroglobin According to the Invention in Normal Tissues and Neuronal Tissue

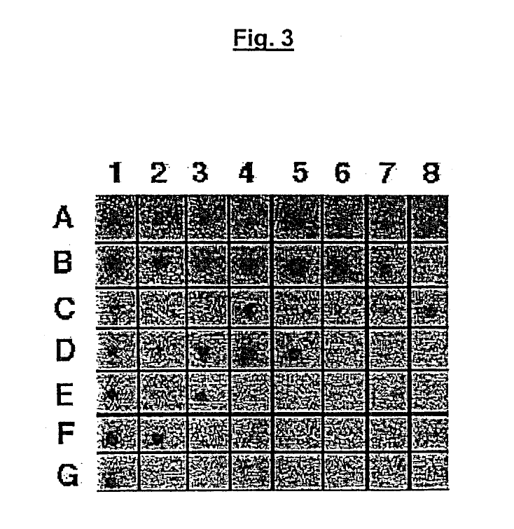

An RNA master Dot-Blot™ (Clontech, Palo Alto, USA) with standardized amounts of RNA from 50 human tissues was hybridized with a radioactively-labelled probe according to the manufacturer's instructions. The hybridization signals were visualized and quantified with the aid of a Fuji BAS-1800 phosphoimager. The fields in FIG. 3 show the following: A1, total brain; A2, amygdala; A3, caudal nucleus; A4, cerebellum; A5, cerebral cortex; A6, frontal lobe; A7, hippocampus; A8, medulla oblongata; B1, occipital pole; B2, putamen; B3, substantia nigra; B4, temporal lobe; B5, thalamus; B6, subthalamic nucleus; B7, spinal cord; C1, heart; C2, aorta; C3, skeletal muscle; C4, colon; C5, bladder; C6, uterus; C7, prostate; C8, stomach; D1, testis; D2, ovary; D3, pancreas; D4, pituitary gland; D5, suprarenal gland; D6, thyroid gland; D7, salivary gland; D8, mammary gland; E1, kidney; E2, liver; E3, small intestine; E4, spleen; E5, thymus gland; E6, peripheral leukocytes; E7, lymph nodes; E8, bone marrow; F1, appendix; F2, lung; F3, trachea; F4, placenta; G1, fetal brain; G2, fetal heart; G3, fetal kidney; G4, fetal liver; G5, fetal spleen; G6, fetal thymus gland; G7, fetal lung.

It turns out that strong hybridization signals of neuroglobin sequences can be detected in brain tissue. Furthermore it turns out that such signals are only very weak in normal tissue.

### EXAMPLE 3

### Preparation and Purification of a Neuroglobin According to the Invention

Murine neuroglobin was cloned into the pET-3a expression vector. For this the coding section of the neuroglobin cDNA was first amplified by PCR. The 5′ PCR primer was equipped with an NdeI cleavage site which provided the translation start codon after insertion into the vector. The 3′ primer contained the stop codon of the cDNA followed by a BamHI cloning cleavage site. The NdeI/BamHI cleaved PCR product was ligated into a pET-3a vector which had also been cleaved with NdeI/BamHI. E. coli BL21(DE3)pLys(F⁻ompT⁻ r⁻_{b} m⁻_{b}) bacteria were transformed with the recombinant plasmid and 1 ml of an overnight culture was used to inoculate 1000 ml LB medium containing 1000 μg/ml ampicillin and 1 mM δ-aminolevulinic acid. The culture was shaken for 6 to 8 hours at 25° C. and 250 revolutions per minute. The expression of neuroglobin was induced by adding 1 mM isopropyl-δ-D-thiogalactopyranoside (IPTG) and the bacteria were allowed to grow for a further 14-18 hours. The bacteria were harvested (20 minute centrifugation at 1000×g), washed with one volume (200 ml) 25 mM Tris-HCl, pH 8.0, 10 mM EDTA, 0.9% glucose and resuspended in 50 mM Tris-HCl, 1 mM EDTA, 0.5 mM DTT, pH 8.0 containing the Roche Complete™Proteinase-inhibitor mixture. The bacteria were lysed by three freeze/thaw cycles in liquid nitrogen and subsequent treatment by ultrasound. Cell debris was removed by centrifugation (1 h, 6000×g). The recombinant neuroglobin was precipitated with 40 to 60% (NH₄)₂SO₄, dialysed overnight against 50 mM potassium phosphate buffer, pH 7.4 containing 1 mM EDTA and 0.5 mM dithiothreitol (DTT) and purified by size exclusion chromatography on a Sephacryl S-200 column (Amersham Pharmacia).

It turns out that it is possible to produce a recombinant protein according to the invention in a highly purified form.

### EXAMPLE 4

### Oxygen Binding Studies

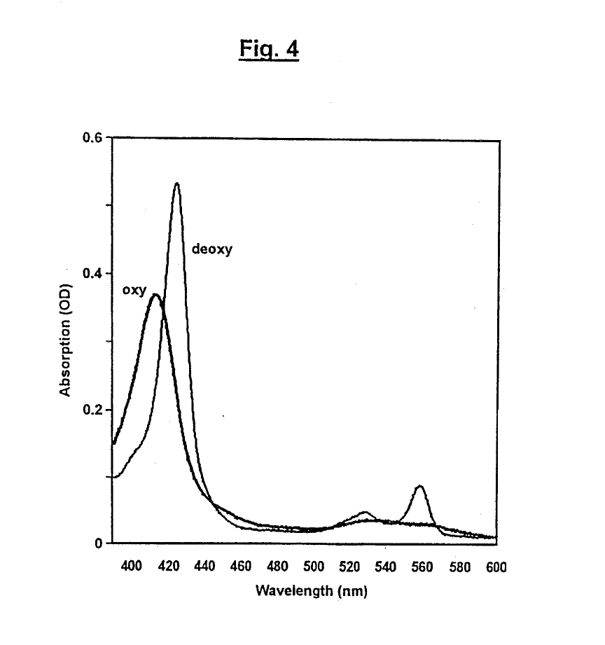

Oxygen binding studies were carried out at 25° C. in 50 mM potassium sulfate, 1 mM EDTA, pH 7.4. The absorption at 424 nm (deoxy maximum) was measured in a Gill cell. Due to the partially reversible oxygenation during the purification process, oxygen binding curves were measured using bacterial supernatant which had previously been concentrated by microfiltration in Centrisat C-4 filters (Sartorius) with an exclusion cut-off of 5000 Da. Supernatants of wild type bacteria which expressed no neuroglobin exhibited no oxygen binding whereas supernatants of recombinant bacteria bound oxygen (cf. FIG. 4). The affinity for oxygen is P₅₀=1.9 to 2.3 torr (typical hemoglobin: P₅₀=26 torr; myoglobin: P₅₀≈1 torr).

This demonstrated that a fusion protein according to the invention binds oxygen with a physiologically relevant affinity and thus fulfils a function in neuronal tissue which is similar to that of myoglobin in muscle.

### EXAMPLE 5

### Preparation and Detection of an Antibody According to the Invention

A recombinant protein according to the invention of example 3 is subjected to a 15% SDS polyacrylamide gel electrophoresis. After staining the gel with Coomassie Brilliant Blue, a ca. 17 kD band is cut out of the gel. The gel pieces are comminuted with 500 μl PBS and the animals are immunized as follows:

Immunization Protocol for Polyclonal Antibodies in Rabbits

ca 50 μg gel-purified recombinant fusion protein in 0.7 ml PBS and 0.7 ml complete or incomplete Freund's adjuvant, respectively, are used per immunization.

day 0: 1st immunization (complete Freund's adjuvant)

day 14: 2nd immunization (incomplete Freund's adjuvant; icFA)

day 28: 3rd immunization (icFA)

day 56: 4th immunization (icFA)

day 80: exsanguination.

The serum of the rabbit is tested in an immunoblot. For this a recombinant protein according to the invention of example 1 is subjected to SDS polyacrylamide gel electrophoresis and transferred onto a nitrocellulose filter (cf. Khyse-Andersen, J., J. Biochem. Biophys. Meth. 10, (1984), 203-209). The Western blot analysis was carried out as described in Bock, C.-T. et al., Virus Genes 8, (1994), 215-229. For this the nitrocellulose filter was incubated for 1 hour at 37° C. with a first antibody. This antibody is the serum of the rabbit (1:10000 in PBS). After several washing steps with PBS, the nitrocellulose filter is incubated with a second antibody. This antibody is a monoclonal goat anti-rabbit IgG antibody (Dianova) coupled to alkaline phosphatase (1:5000) in PBS. After a 30 minute incubation at 37° C., it is washed several times with PBS and subsequently the alkaline phosphatase detection reaction is carried out using developer solution (36 μM 5′ bromo-4-chloro-3-indolyl phosphate, 400 μM nitroblue-tetrazolium, 100 mM Tris-HCl, pH 9.5, 100 mM NaCl, 5 mM MgCl₂) at room temperature until bands become visible.

Immunization Protocol for Polyclonal Antibodies in the Chicken

40 μg gel-purified recombinant protein in 0.8 ml PBS and 0.8 ml complete or incomplete Freund's adjuvant, respectively, were used per immunization.

day 0: 1st immunization (complete Freund's adjuvant)

day 14: 2nd immunization (incomplete Freund's adjuvant; icFA)

day 28: 3rd immunization (icFA)

day 56: 4th immunization (icFA)

Antibodies are extracted from egg yolk and tested in the Western blot. Polyclonal antibodies according to the invention are detected.

Immunization Protocol for Monoclonal Antibodies in the Mouse

12 μg gel-purified recombinant human neuroglobin in 0.25 ml PBS and 0.25 ml complete or incomplete Freund's adjuvant, respectively, is used per immunization; during the 4th immunization the fusion protein is dissolved in 0.5 ml (without adjuvant).

day 0: 1st immunization (complete Freund's adjuvant)

day 14: 2nd immunization (incomplete Freund's adjuvant; icFA)

day 28: 3rd immunization (icFA)

day 84: 4th immunization (PBS)

day 87: fusion

Supernatants of hybridomas are tested in the Western blot. Monoclonal antibodies according to the invention are detected.

1. A protein named “neuroglobin” with a globin-like structure which does not belong to the family of hemoglobins and myoglobins of vertebrates, is derived from neuronal tissue, binds oxygen and stores and transports oxygen in the said tissue.

2. A protein according to claim 1, characterized in that it has a molecular weight of about 17,000.

3. A protein as according to claim 1 or 2, characterized in that it comprises the amino acid sequence of SEQ ID No. 1 or SEQ ID No. 2 or an amino acid sequence which differs therefrom by one or more amino acids.

4. A protein according to any of the claims 1 to 3, characterized in that the DNA sequence which encodes the protein, hybridizes with the DNA sequence according to SEQ ID No. 1 or SEQ ID No. 2.

5. A neuroglobin gene having the genomic nucleic acid sequence of SEQ ID No. or a gene which differs therefrom in one or more base pairs.

6. A nucleic acid coding for a protein according to any of the claims 1 to 4 comprising: (a) the DNA of SEQ ID No. 3 or 4 or a DNA or RNA which differs therefrom by one or more base pairs where the latter DNA or RNA hybridizes with the DNA of SEQ ID No. 3 or 4, or (b) a DNA related to the DNA of (a) via the degenerate genetic code.

7. A ribozyme, characterized in that it is complementary to a nucleic acid sequence according to claim 5 or 6 and specifically binds to the cDNA transcribed from this nucleic acid sequence and can cleave this DNA, which results in a reduction or inhibition of the synthesis of the protein coded by this nucleic acid sequence.

8. An antisense RNA, characterized in that it is complementary to a nucleic acid sequence according to claim 5 or 6 and can specifically bind to this DNA, which results in a reduction or inhibition of the synthesis of the protein coded by this nucleic acid sequence.

9. An expression vector containing a nucleic acid sequence according to claim 5 or 6 or a nucleic acid sequence coding for the ribozyme according to claim 7 or the antisense RNA according to claim 8.

10. A host cell, transformed with the expression vector according to claim 9.

11. An antibody which binds to a protein according to one of the claims 1 to 4 or a fragment thereof.

12. A pharmaceutical preparation containing a protein according to one of the claims 1 to 4 or a ribozyme according to claim 7 or an antisense RNA according to claim 8 or an antibody according to claim 11, and optionally pharmaceutically acceptable auxiliary substances and carriers.

13. An in vitro diagnostic method for the detection of the expression of a protein according to claim 1 to 4, in which a sample is contacted with at least one nucleic acid according to claim 5 or 6 and/or at least one ribozyme according to claim 7 and/or at least one antisense RNA according to claim 8 and/or at least one antibody according to claim 11 and then it is determined directly or indirectly whether the concentration, length and/or sequence of the neuroglobin or the DNA or mRNA which encodes this neuroglobin, differs from a control sample.

14. A diagnostic kit for carrying out the method according to claim 13 which contains at least one component which has a nucleic acid according to claim 5 or 6 and/or at least one ribozyme according to claim 7 and/or at least one antisense RNA according to claim 8 and/or at least one antibody according to claim 11.

15. A use of a protein according to one of the claims 1 to 4, a nucleic acid according to claim 5 or 6, a ribozyme according to claim 7, an antisense RNA according to claim 8 and/or an antibody according to claim 11 for the manufacture of a medicament for the therapy of diseases of the nervous system, stroke, neuronal oxygen deficiency and tumours of neuronal tissue.
